(19) United States
(12) Patent Application Publication
(10) Pub. No.: US 2010/0297047 A1
(43) Pub. Date: Nov. 25, 2010

(54) ESTERS OF GLYCEROL AND THEIR USES IN COSMETIC AND PHARMACEUTICAL APPLICATIONS

(75) Inventors: Giacomo Santus, Agrate Brianza (IT); Laura Barberi, Agrate Brianza (IT); Cristina Schubert, Agrate Brianza (IT)

Correspondence Address: YOUNG & THOMPSON, 209 Madison Street, Suite 500, Alexandria, VA, 22314, US

(73) Assignee: BIOPHIL ITALIA S.P.A., Agrate Brianza (MI) (IT)

(21) Appl. No.: 12/740,009

(22) PCT Filed: Oct. 28, 2008
(86) PCT No.: PCT/EP2008/009097

§ 371 Date: Jul. 30, 2010

(30) Foreign Application Priority Data
Oct. 29, 2007 (IT) MI2007A002081

(51) Int. Cl. A61K 8/92; A61K 47/14; A61Q 19/10; A61Q 5/02; A61Q 19/00; A61Q 17/04; A61Q 5/00; A61Q 1/10; A61Q 1/04; A61Q 1/06

(52) U.S. Cl. 424/60; 514/786; 424/70.1; 510/513; 510/158; 424/70.7; 424/64

(57) ABSTRACT

The invention provides mixtures of tri-, di-, and mono-glycerides obtained by esterification of fatty acids from vegetal oils such as olive oil with glycerine, their production process and their use in cosmetic and pharmaceutical preparations, in particular as vehicle or solubiliser for active ingredients.

The present invention refers to mixtures of tri-glycerides, di-glycerides and mono-glycerides obtained by esterification of fatty acids from vegetal sources, such as olive, with glycerine, their use in cosmetic and pharmaceutical preparations, in particular for the skin treatment, optionally in combination with other active substances. Any vegetal seed used in the vegetal oil industry (e.g. sunflower, corn, rapeseed) may be a source of materials successfully employed in this production process, as well as waste fractions from the same vegetal oils industry.

### BACKGROUND OF THE INVENTION

Tri-glycerides, also commonly called vegetal oils, are natural products which are very important for cosmetics formulations, and for products with topic use in general. Nature has made available a great variety of these materials, potentially useful for the preparation of products for body care, whose chemical composition, and consequently their properties, depend on the vegetal source, e.g. seed, fruit or tree from which oils are extracted, and from the process used to extract the oil.

Vegetal tri-glycerides (or triacylglycerols, or fats and oils) are natural esters formed by glycerine with fatty acids of high molecular mass. This is what happens in nature, both on the tree and seeds, through a precise sequence of enzymatic reactions able to create in the fruit or in the seed a certain amount of tri-glyceride, called fat and/or oil, which represents the energetic source for the seed during its germination phase.

The above described esterification reaction can happen in a very short time, few hours respect to the several months of duration of the natural one, by adopting special technological conditions and by using fatty acids and glycerine, the two fundamental components of the esterification reaction.

Vegetal oils have been used for topic applications since long for both curative and aesthetic purposes.

Olive oil shows peculiar characteristics, for which it can be easily used in “natural” cosmetic formulations, prepared for the application on human skin: its composition in fatty acids is similar to the human sebum; olive oil in addition adsorbs UV radiations between 220 and 300 nm. Thanks to the high physiological compatibility with the human skin, the olive oil is able to restore the bactericide barrier of the epidermis, it helps the moisturization of the skin, is lenitive, emollient and can be useful for the treatment of burns, insect bites, and itch, as well as for eczema and dermatitis. In addition, thanks to the high percentage of mono-unsaturated fatty acids, olive shows increases of the coetaneous permeability, so that it can act as vehicle to deliver the active ingredients into the deeper layers of the skin.

In olive oil, as in many fatty raw materials of vegetal origin, the saponifiable fraction (essentially tri-glycerides) accounts for ca. 98%, being the remaining 1-1.5% unsaponifiables, like in the case of extra virgin oil used for food consumption. The unsaponifiable fraction is constituted by a large number of micro-components which have the common characteristics of not forming soap if treated by a concentrated strong base at high temperature (NaOH or KOH). In the natural olive oil these components are: hydro-carbons, paraffins, waxes, alcohols, sterols, colour pigments, oil-soluble vitamins, polyphenols.

The unsaponifiable fraction contained in the oils—very important from a nutritional point of view, and representing per se an optimal active ingredient for cosmetic and dermatologic formulations—can cause instability and inconvenients in skin feel when formulating cosmetic products containing the vegetal oil (for instance in the case of olive oil), due both to organoleptic aspects (strong odour and colour), and to the different physical chemical behaviours (e.g. solubility, solidification point) of its components.

Triglycerides, diglycerides and monoglycerides of both vegetal and animal origins, are important raw materials in cosmetics. The most requested characteristics of these products are the absence of organoleptic characters, the ease of use, the preference for liquid products. The latter physico-chemical characteristic is typical for oils with short-chain or highly unsaturated fatty acids: these are more valuable and allow more attractive and pleasant formulations for topical applications. On the other hand, they are more expensive.

The widely used products are blends of short chain tri-glycerides (e.g. caprylic/capric triglycerides) or oleic/linoleic/linolenic poly-glycerides, oleic/linoleic poly-glyceride/tri-glycerides, oleic/palmitic/lauric/miristic/linoleic tri-glycerides. These products are normally obtained by esterification of synthetic fatty acids with glycerine, in order to obtain tri-olein and/or tri-linolein, and saturated fatty acids esters, which are normally used in the surfactants industry, in rubbers and elastomers.

### DESCRIPTION OF THE INVENTION

Object of this invention is a mixture of tri-glycerides, di-glycerides and mono-glyclerides—hereafter indicated as “mixtures containing esters of glycerol with fatty acids”—obtained from vegetal oils normally used in the food industry such as virgin olive oil, sunflower oil, rapeseed oil and other sources of fatty oils, by means of a process improving the physico-chemical, rheological, formulative, and dermatological properties of the product and thereby its use in cosmetic and pharmaceutical applications. In particular the following characteristics are improved: thermal and oxidative stability, ease of emulsification and/or dispersion, solubilisation, skin-feel and cutaneous affinity.

The process according to the invention comprises the following steps:

a) neutralisation of the clear vegetal oil (for instance, virgin olive oil) by NaOH, preferably using NaOH at 20 Bè (corresponding to 14.4 kg of caustic soda for 100 kg of aqueous solution) with consequent formation of soap-like paste;

b) acid hydrolysis of the soap-like pastes with consequent formation of a fatty phase containing free or esterified fatty acids with glycerol, and an aqueous phase, containing mucilage, impurities and phospholipids;

c) separation of the fatty phase from the liquid phase;

d) distillation of the fatty acids contained in the fatty phase at a pressure from 1 to 3 mbar;

e) esterification of the fatty acids distilled with glycerine, using a glycerine to fatty acids ponderal ratio of 1-2/10, at a temperature between 200-220° C. and pressure from 1 to 3 mbar;

f) neutralisation of the obtained product to eliminate the residual acidity;

g) decolouring, deodorizing and optional winterization of the product by means of direct vapour input at a pressure from 1 to 3 bar.

In one embodiment of the invention, the residual fraction not distilled at step d), containing neutral oil, is hydrolysed to form fatty acids and an aqueous phase containing glycerine; the aqueous phase is separated from the fatty acids and these are processed according to the steps d)-g). Preferably the hydrolysis of the residual fraction is carried out at a pressure around 30 bar and a temperature around 230° C.

In another embodiment of the invention, the water phase containing glycerine is concentrated, centrifuged and then distilled under deep vacuum (1-4 mbar) at a temperature of 140-170° C. in order to prepare a glycerine with a high degree of purity, which is subsequently used for the esterification of the fatty acids according to step e).

Preferably, glycerine and distilled fatty acids have a purity of at least 99.5%.

In another embodiment of the invention, the product obtained at the end of step g) is distilled at a temperature between 220 and 280° C., operating at a maximal pressure of 0.001 mbar through a thin film evaporator and/or short path distillator, with an average resident time of the product ranging from 30 seconds up to 2 minutes, to prepare distilled fractions enriched in diglycerides with purity exceeding 90% or triglyceride residual fractions with purity exceeding 95%.

Another aspect of the invention refers to a mixture of esters of glycerol with fatty acids, obtained by the process described here above, and to its use in medical and cosmetic applications. According to a preferred embodiment, this mixture is characterised by the following fatty acids % composition, where olive oil and sunflower oil are used as the starting materials:

| | Olive Oil | Sunflower Oil |
|---|---|---|
| Palmitic | 5-12% | 5-7.6% |
| Palmitoleic | 1.5-2.5% | 0-0.3% |
| Stearic | 1.5-4% | 2.7-6.5% |
| Oleic | 65-80% | 14.0-39.4% |
| Elaidinic (trans-oleic C18 n-9) | 0-0.4% | — |
| and petroselinic (cis C18 n-12) | | |
| Linoleic | 10-20% | 48.3-74.0% |
| Linolenic | 0.5-1% | 0-0.3% |
| Arachic | 0.3-0.7% | 0.1-0.6% |
| Eicosenoic (gadoleic) | 0.2-0.6% | 0-0.3% |
| Beenic | 0.1-0.3% | 0.3-1.5% |
| Erucic | — | 0-0.1% |
| Lignoceric | 0.1-0.3% | 0-0.5% |
| Miristic | — | 0-0.2% |
| Lauric | — | 0-0.1% |
| Heptadecanoic | — | 0-0.2% |
| Heptadecenoic | — | 0-0.1% |

preferably the mixture contains from 25 to 80% wt triglycerides, from 0 to 50% diglycerides and from 0 to 25% monoglycerides.

In a further aspect the invention refers to a cosmetic or pharmaceutical composition containing a mixture of esters of glycerol with fatty acids as herein described. This composition will be preferably produced in form of cream appearance, gel, unguent, lotion, emulsion o/w and w/o, detergent, shampoo, bath foam, gel and shower foam, liquid soap, stick, make up products, cosmetic oil.

A further aspect of the invention refers to a method for the cosmetic treatment of the skin, hair, eyes contour, lips, which comprises the application of a composition according to the invention on the interested part of the body.

A yet further aspect of the invention refers to the use of a mixture of esters of glycerol with fatty acids, as here defined, as a carrier or solubiliser for active ingredients or as excipients for the preparation of a cosmetic or pharmaceutical composition.

### DETAILED DESCRIPTION OF THE INVENTION

1. Method for the Preparation of Fatty Acids and Glycerine

During the refining cycle of the vegetal oil (for instance olive oil) to alkalis (“hot phase”), in the neutralization phase the free acidity (FFA) is eliminated by means of a caustic solution of NaOH with the formation of soaps (soap-stock) and consequent dragging of neutral oil (triglyceride) that forms during the centrifuge process of the soaps-oil mixture present in the neutralised olive oil. The soap-like paste is then subjected to acidic hydrolysis, by which acidic oils (oleins, 65-75% FFA in case of olive oil), neutral oil and a mucilage-like interphase of phospholipids, impurities and pigments are obtained.

After washing and purification of the above mixture, a fatty phase is obtained which is constituted by oleins and neutral oil and which is then subjected to:

1. distillation under deep vacuum of the fatty acids contained in the “acidic oils” (oleins), obtaining a product of very high purity and white in colour, ready for the successive esterification phase

2. the remaining neutral oil fraction (30-40%) is subjected to hydrolytic cleavage in excess of water, at a pressure of around 30 bar and at the temperature of 230° C. In these technological conditions a catalyst is not used and from each triglyceride molecule a molecule of glycerine and three molecules of fatty acids are obtained, which are subjected to the distillation reported above.

From the hydrolysis reaction, together with the fatty acids, as above reported, the glycerine waters are obtained, which are subjected to concentration, distillation and depuration to obtain glycerine with a purity of 99.5%.

When the pure fatty acids are obtained from the by-products of the refining process (soap like pastes in the neutralization process) of olive oil, the same % composition present in the original pure product is obtained.

2. Esterification

The esterification is carried out by adding glycerine to the fatty acids in a 1-2:10 ratio under the following process conditions:
deep vacuum from 1 up to 3 mbar to help the reaction and protect the product from oxidation
operative temperature around 200/220° C., increasing the contact between fatty acid and glycerine by means of an efficient stirring,

(yields around 94-95%). The weight ratio reported above (molar excess of glycerine) is optimal to give high yield of diglycerides and triglycerides, unexpected from the stoichiometry of the reaction.

The esterified crude vegetal oil obtained is then subjected to the neutralization phase to eliminate the residual acidity, then is decoloured and finally is deodorised under deep vacuum, with the use of direct vapour, in order to prepare an esterified oil with good qualitative characteristics. Using olives as vegetal source, a product of low acidity is obtained (<0.3%), with a low content of unsaponifiable matter (<200 ppm in the finished product). This product is similar to the olive oil that can be commercialised as refined oil for technical use or for food preparation. As reported above the esterified olive oil shows a high content of diglycerides as compared to the natural oil and a different distribution of the fatty acids in the three carbons of glycerine.

The improvement of the production process described above, including the winterization step which contributes to a better stability, a higher liquidity and a lower viscosity, allows to prepare a product with both a high purity and pleasant feel, chemical-physical and rheological conditions adequate for the use in cosmetic and dermatological applications.

3. Separation/Enrichment of Triglycerides and Diglycerides

The product obtained at the end of the process described in the previous sections, is a mixture of triglycerides (the main fraction, 50-80%) diglycerides (20-50%) and a minor quantity of monoglycerides.

These are high boiling products, which by the standard distillation techniques are not easy to separate due to the high temperature needed (above 500° C.) which causes technological hurdles and above all a high degree of decomposition of the product during this process. In particular, the decomposition increases exponentially with the temperature and linearly with the residence time in the distillator.

Diglycerides have been separated with success from the triglycerides using a Thin film evaporation (short path distillation), which permits to distil at lower temperatures (220-280° C.) working under deep vacuum (up to 0.001 mbar), with a product average residence time of 1 minute. Adjusting with high precision the distillation temperature and the deep vacuum conditions, distilled fractions enriched in diglycerides having purity above 90% are obtained; or mixtures of diglycerides and triglycerides can be distilled, leaving residues with triglycerides fractions above 95%.

4. Use in Cosmetics and Dermopharmaceutics

The products obtained as described in the previous sections, can be used in cosmetics and in pharmaceutics.

The products object of the invention can be used either as a lipophilic fraction in cosmetic formulations or as a vehicle/solubiliser of active ingredients in cosmetic/pharmaceutical formulations. For both uses, the most suitable mixtures of glycerides/triglycerides can be chosen, depending on the requested characteristics: hydrophilic/lipophilic balance, solubilising property of active ingredients based on affinity, compatibility with others ingredients in the formulation, final texture required for the formulation.

For the cosmetic/pharmaceutical uses, the products of the invention will be formulated optionally in combination with other dermatologically active substances Suitable formulations include all the cosmetic products and dermo pharmaceutics, such as creams, gels, pastes, unguents, lotions, emulsions o/w and w/o, detergents and toiletries (shampoos, bath foam, gels and shower foam, liquid soaps), sticks, make up products, cosmetic oils. The principle and the methods for the preparation of these formulations are known to the skilled artisans and are described for example in Remington's Pharmaceutical Science, 17° edition, Mack Publishing Company, Easton PS, 1985.

In addition to the products of the invention, the formulations can contain other biologically active ingredients and excipients, such as surfactants, emollients, emulsifiers, solvents, humectants, promoters of percutaneous adsorption, and in general all the typologies of ingredients which are well known to the formulators of cosmetics and pharmaceuticals.

The quality and the type of the triglycerides/diglycerides mixtures will vary depending on the destination of use and on the specific formulation.

In general, when used as lipidic component in cosmetic/dermatological preparations, the amount of mixture can range from 1% to 98% depending on the kind of formulation; when the product is used as a vehicle for the dissolution of active ingredients, its quantity will depend on the concentration of the active ingredients, on its application dosage, and on its physico-chemical and solubility properties.

Due to the higher percentage of diglycerides compared to natural vegetal oil, these products show a higher polarity, featuring a higher miscibility and solubilising characteristics or affinity with polar substances, properties which are practically absent in the natural oil. This permits their use in many different formulations or as a legant in the formulations, overall helping their emulsification.

In addition, mixtures enriched in triglycerides will be preferably used with lipophilic active substances, with the advantages explained afterwards.

Diglycerides-enriched mixtures, due to the increased hydrophilic character, give these products a higher solubilising and emulsifying property, as compared to the basic oils or fats.

### EXAMPLES

The following formulations of olive oil glycerides have been prepared (for each formulation the quantity of the mono-di and triglycerides mixture is indicated); the advantages of the mixture of glycerides are reported in comparison with commonly used oils.

Very similar mixtures of glycerides can be obtained from other common vegetal sources such as sunflower, rapeseed, and others, so similar properties and performances can be expected by most of the common vegetal oils.

1. face and body cream: 0.5-20%; suncare cream: 0.5-20%

Oil with optimal cutaneous affinity, thanks to the lipidic composition from olive oil, similar to sebum. Better thermal and oxidation stability, lighter and pleasant touch in respect to other natural vegetal oils, easy emulsification.

2. lipsticks: 5-30%

3. glosses: 2-30%

Alternative to the use of castor oil or other vegetal oils which are normally used for this application (stick). Good dispersion capacity for pigments. Good thermal stability, more pleasant odour and flavour (which is highly important for these applications), technically edible oil.

4. Foundation in cream form: 0.5-20%

Oil with excellent cutaneous affinity, thanks to the lipidic composition from olive oil. Better thermal and oxidation stability, lighter and more pleasant touch with respect to the usually employed vegetal oils (palm). Easy emulsification and good stabilising property.

5. Poured foundation: 0.5-20%

Oil with excellent cutaneous affinity and which is not sticky when applied onto the skin (not-sticky fat). Better thermal and oxidation stability, lighter and more pleasant touch as compared to the other vegetal oils. Easy emulsifying and handling during the preparation.

6. Mascara: 0.5-30%

Imparts good spreadability and adhesion of the product to the eyelash. Good compatibility with the components of these formulations.

7. Shampoo: 0.5-5%

8. Hair mask: 0.5-5%

9. hair restructuring oil: 5-80%

restructuring properties of the hair, lighter touch in comparison to the other oils which are normally used in this application (almond, wheat germ, argan, . . . ), easy handling; higher affinity towards water and hydrophilic components affinity due to the diglyceric fraction

10. Bath cream: 0.5-20%

Excellent emollient property and capability to reduce the irritation caused by the primary anionic surfactants

11. Body butter; 0.5-20%

Oil with excellent cutaneous affinity, thanks to the lipidic composition of the olive oil. Improved thermal and oxidation stability, lighter and more pleasant touch in comparison to the other vegetal oils. Improved feeling when these products are applied onto the skin, improved binding capacity of the components.

12. Hair waxes: 0.5-20%

Restructuring hair property, light touch

13. Suncare oils: 20-50%

Excellent solvent property for UV filters, pleasant touch on the skin.

14. Massage Oil: 20-90%

Good spreading property

Use as vehicle of active ingredients

Improved formulations have been prepared by solubilising lipophilic ingredients, which are known for their interesting cosmetic activity but also for their difficult usage due to their poor solubility and/or dispersion into common cosmetic matrixes.

In particular, we have optimised:

1. A Ceramide-3 Dispersed into a Mixture of Olea europea Triglycerides/Diglycerides 75/25

Ceramides are major components (roughly 40%) of the lipidic layers of the epidermis barrier, which drive the balance between hydration and loss of water, having the important role of “cement” between the corneocytes, i.e. the brick cells forming the dermal layers. The high difficulty for the application of ceramides in cosmetics is their poor solubility: in order to solubilise them into esters, a heating step above 90° C. is normally required with possible damage of the ceramides.

The use of triglycerides/diglycerides mixture as a solubiliser has allowed the solubilisation of ceramide-3 also at high concentration (up to 50%) without excessive and long time heating steps, A liquid ingredient, easy to use by simple addition into the lipid phase of the cosmetic formulation, has been obtained so far. Clinical tests have demonstrated the efficacy of this mixture to improve the elasticity of the skin and the reduction of the wrinkles depth (smooth skin)

2. A Beta-Sitosterol Dispersed in the Olea europea Triglycerides/Diglycerides 75/25 mixture 75/25

Beta sitosterol (24-ethyl-delta-5.cholesten-3 beta-ol or Stigmastan-5-en-3 beta ol—CAS 83-46-5) is an active ingredient used both in the pharmaceutical sector, to treat the hyper-cholesterol and the prostatic adenoma, and in cosmetic formulations for its excellent emulsifying property. Its dissolution into the mixture of this invention has permitted to get easy-to-use ingredients, with advantages similar to the one described above for ceramides. Also in this case the clinical evaluations have demonstrated the efficacy of this association.

3. A high molecular weight sericin dispersed in the Olea europea triglycerides/diglycerides 75/25 mixture.

Sericin is one of the two families of proteins constituting the pure silk secreted by the silkworm glands: its dimensions vary from 60 up to 500 kDa. Recently, several important properties of sericin have been reported in the scientific literature, regarding its affinity with the skin and human hair, as well as a strong attachment to the fibroblasts of human skin cultivation. A sericin with high molecular weight, showing interesting cosmetic traits, but difficult to use due to the large molecular dimensions (microcrystalline solid material with very poor solubilisation) has been successfully dissolved in mixtures of triglycerides/diglycerides from olive in significant quantities and more easily with respect to other commonly used vehicles or solubilisers. These did not give the same results due to the double nature of this protein (polarity from the peptidic bonds, high lipophilic character from the great dimensions yielding a high degree of the folding of this protein).

Differences Between Natural Vegetal Oil Such as Olive Oil, Sunflower Oil, Rapeseed Oil, and Respective Oil Derivatives According to the Invention

The main differences between the products objects of the invention and the olive oil obtained by natural bio-synthesis are the following:

1. in the natural vegetal oil the diglycerides are contained in low quantities, for instance in the olive oil the diglycerides are not above 3%; in the products high diglyceride amounts (from 20 up to 30%) are found, together with small percentages of monoglycerides, which result statistically to be the uncompleted products of the esterification reaction. The diglycerides, having a free hydroxyl group, can bind to polar compounds, forming stable emulsions. As already mentioned, from the original mixture obtained from the esterification process, isolated mixtures, enriched more in triglycerides or in diglycerides can be isolated;

2. in the diglycerides of the invention the position of the two fatty acids with respect to glycerine are different with respect to the naturally occurring dyglicerides; for instance, the diglycerides of the esterified olive oil account for 33% in position 1.2 and 67% in position 1.3 (which is the most stable form of the diglyceric isomeric structure); in the olive oil obtained by natural bio-synthesis, the positioning is the opposite, 90% in position 1.2 and during the time, for natural rearrangement, less then 50% are in position 1.2 and more then 50% in position 1.3, with a maximum absolute amount of 3%.

3. in the product according to the invention the fatty acids are positioned (esterified) randomly in the 3 positions of glycerine, while in the natural oils the saturated fatty acids are positioned quite exclusively in correspondence of the positions 1 and 3 of glycerol, while the position 2 is almost entirely occupied by unsaturated fatty acids.

4. in the natural olive oil the double bonds are separated and with a cis geometry; due to the hard conditions of the hydrolysis process and subsequent re-esterification, in the product of the invention a small percentage has an isomeric trans structure and/or migrates to form conjugated dienes.

5. finally, in the esterified oils, due to the sequence of the hydrolysis, distillation and esterification reactions, only around 100 ppm of non-lipophilic compounds are observed, as opposed to the much higher quantities observed in the natural oil (for instance, 13000 ppm are is present in the natural olive oil). The unsaponifiable fraction is therefore practically absent.

### Application Examples

The examples that follow are showing in better detail the applications of this invention in cosmetic and dermo-pharmaceutical formulations. In particular, formulations containing mixtures of the glycerides according to the invention are reported.

1-24 Hours W/O Cream—Ref. 0025

| Phase | Components | INCI name | % |
|---|---|---|---|
| A | Surface-active | PEG-8 Stearate (and) Cetearyl | 10.00 |
| | substances/ | Ethylhexanoate (and) Isopropyl | |
| | emulsifiers | Myristate (and) Glyceril Stearate (and) | |
| | DERMAPHYL | Stearyl Heptanoate (and) Cetyl Alcohol | |
| | AE 102 | (and) Butyl Stearate (and) Cetyl | |
| | | Palmitate (and) Sorbitan Sesquioleate | |
| | | (and) Stearic Acid (and) Aqua (and) | |
| | | Potassium Hydroxyde (and) BHA | |
| | Oliphyl TD 7525 | Olea Europaea (OLIVE) Diglyerides | 8.00 |
| | | and Triglycerides | |
| B | Water | Aqua | 81.50 |
| | Active ingredient | Yaluronic Acid | 0.10 |
| | Phylcare | | |
| | Hyaluronate HW | | |
| | Preservative | Preservative | q.b. |
| | Disodium EDTA | Disodium EDTA | 0.10 |
| | Flavour | Parfum | 0.30 |

Preparation

1. mix the components of phase (A), heat at 75° C. under stirring;

2. prepare phase (B) and heat at 75° C.; the components should be added in the order indicated;

3. activate the turboemulsifier and add phase (A) to phase (B); turboemulsifier in action for 20 minutes;

4. cool to room temperature under mild stirring.

2—“Intensive” W/O Cream—Ref. 0026

| Phase | Components | INCI name | % |
|---|---|---|---|
| A | Surface-active | PEG-8 Stearate (and) Cetearyl | 10.00 |
| | substances/ | Ethylhexanoate (and) Isopropyl | |
| | emulsifiers | Myristate (and) Glyceril Stearate (and) | |
| | DERMAPHYL | Stearyl Heptanoate (and) Cetyl Alcohol | |
| | AE 102 | (and) Butyl Stearate (and) Cetyl | |
| | | Palmitate (and) Sorbitan Sesquioleate | |
| | | (and) Stearic Acid (and) Aqua (and) | |
| | | Potassium Hydroxyde (and) BHA | |
| | Oliphyl TD 7525 | Olea Europaea (OLIVE) Diglyerides | 8.00 |
| | | and Triglycerides | |
| | Vitamin E | Tocopheryl Acetate | 0.50 |
| B | Demineralized | Aqua | 80.00 |
| | water | | |
| | wetting Panthenol | Panthenol | 0.50 |
| | | Sericin | 0.10 |
| | Preservative | | q.b. |
| | Disodium EDTA | Disodium EDTA | 0.10 |
| C | Active ingredient | Beta-glucan | 0.50 |
| | Flavour | Parfum | 0.30 |

Preparation

1. mix the components of phase (A), heat at 75° C. under stirring;

2. prepare phase (B) and heat at 75° C.; the components should be added in the order indicated;

3. activate the turboemulsifier and add phase (A) to phase (B); turboemulsifier in action for 20 minutes;

4. cool to room temperature under mild stirring, then add phase (C).

3—Bath-Shower Cream—Ref. 0037

| Phase | Components | Denominazione INCI | % |
|---|---|---|---|
| A | Water | Aqua | 41.45 |
| | Conditioning | Hydrohypropyl Guar | 0.30 |
| | Jaguar C162 | Hydroxypropyltrimonium Chloride | |
| B | Surface-active | Sodium Trideceth Sulfate (and) Sodium | 40.00 |
| | substances | Lauroamphoacetate (and) Cocamide | |
| | | MIPA (and) Water | |
| C | Oliphyl TD7525 | Olea Europaea (OLIVE) Diglyerides | 15.00 |
| | | and Triglycerides | |
| | Antioxidant | BHT | 0.05 |
| | BHT | | |
| | Perfume | Perfume | 0.60 |
| D | Sodium chloride | Sodium Chloride | 2.00 |
| E | Preservatives | Preservatives | q.b. |
| F | Acidity corrector | Citric Acid | q.b. |

Preparation

1. mix the components of phase (A);

2. add phase (B) to phase (A) under stirring and continue mixing for 30 min;

3. add one by one the components of phase (C);

4. Add, in the order, phase (D), phase (E) and adjust pH to 5.0-6.0 with phase (F).

4—Restructuring Shampoo—Ref. 0039

| Phase | Components | INCI name | % |
|---|---|---|---|
| A | Wetting agent | POLYSORBATE-20 | 2.0 |
| | Perfume | PARFUM | 0.60 |
| | OLIPHYL 7525 | Olea Europaea Diglycerides and | 0.50 |
| | | triglycerides | |
| | Surface-active substance | SODIUM COCOAMPHOACETATE | 12.0 |
| | Surface-active substance | SODIUM LAUROYL GLUTAMATE | 8 |
| | Surface-active substance | COCAMIDOPROPYL BETAINE | 6 |
| | Surface-active substance | LAURETH-2 | 1.20 |
| | Additional surface-active | COCOGLUCOSIDE, GLYCERYL | 0.50 |
| | substance | OLEATE | |
| | Active ingredient | POLYQUATERNIUM-7 | 2.0 |
| B | Water | AQUA | q.b. 100 |
| | EDTA | EDTA | 0.15 |
| | rheological agent | POLYACRYLATES | 5.0 |
| | polyacrylate | | |
| | Active ingredient | PANTHENOL | 0.3 |
| | Panthenol | | |
| | Surface-active substance | SODIUM LAURETH-SULPHATE | 20 |
| C | Preservative | Preservative names | q.b. |
| D | acidity corrector | CITRIC ACID | q.b. a pH |
| | | | 6.2-6.4 |

Preparation

1. mix the components of phase (A) by adding the ingredients in the order indicated;

2. prepare phase (B) by adding the ingredients in the order indicated; neutralize with NaOH until the solution is completely clear;

3. add phase (A) to phase (B);

4. add, in the order, phases (C) and (D) adjusting the pH and viscosity.

5—Contour Eyes Fluid—Ref. 0002

| Phase | Components | INCI name | % |
|---|---|---|---|
| A | Osmotic water | AQUA | q.b. 100 |
| | EDTA | EDTA | 0.10 |
| | Glicerol F.U. | GLYCERIN | 3.80 |
| | Active ingredient | ALLANTOIN | 0.30 |
| | Active ingredient | HYALURONIC ACID | 0.20 |
| | Active ingredient | THIAMINE CHLORIDRATE | 0.10 |
| | Emulsifying ester | SUCROSE PALMITATE | 2.50 |
| B | Demineralized | AQUA | 0.50 |
| | water | | |
| | cetearyl alcohol | CETEARYL ALCOHOL | 0.20 |
| | Olyphil TD7525 | Olea Europaea Diglycerides and | 8.0 |
| | | triglycerides | |
| | Active ingredient | THEOBROMA GRANDIFLORUM | 2.0 |
| | Active ingredient | BETAGLUCAN | 2.0 |
| | Active ingredient | BETA SITOSTEROL | 0.50 |
| | Active ingredient | BISABOLOL | 0.10 |
| C | ISOCIDE SCM | PROPYLENE GLYCOL, | 2.50 |
| | | METHYLPROPANEDIOL, | |
| | | CHLORPHENESIN, SORBIC ACID | |
| | Vegetal active | QUERCUS ROBUR EXTRACT | 0.50 |
| | ingredient | | |
| | PHYLCARE | | |
| | MERISTEMIN | | |

Preparation

1. mix the components of phase (A)

2. disperse the polymer by means of the turboemulsifier

3. prepare phase (B) heating at 75° C.

4. pour phase (B) in (A), activating the turbine for some minutes

5. cool and add phase (C) at 40° C.

6—Sun Care Protective Cream (Theoretical SPF 8)—Ref

| Phase | Components | INCI name | % |
|---|---|---|---|
| A | Emulsifier | PEG-6 STEARATE, GLYCOL | 5 |
| | DUB 1632 | STEARATE, PEG-32 STEARATE | |
| | cetearyl alcohol | CETEARYL ALCOHOL | 0.20 |
| | Gliceryl monostearate | GLYCERYLMONOSTEARATE | 2.5 |
| | DUB PC ester | CETYL PALMITATE | 1 |
| | LIPOPHYL CUPUACU | THEOBROMA GRANDIFLORUM | 2 |
| | BUTTER | (SEED BUTTER) | |
| | OLIPHYL 7525 | Olea Europaea Diglycerides and | 10 |
| | | triglycerides | |
| | Silicone | TRIMETHYL SILOXISILICATE | 0.5 |
| | Silicone | BIS-HYDROXYETHOXYPROPYL | 2 |
| | | DIMETHICONE | |
| | Sunscreen | ETHYLHEXYL | 5 |
| | | METHOXYCINNAMATE | |
| | Sunscreen | BUTYLMETHOXYDIBENZOYLMETHANE | 0.5 |
| | Antioxidant | | QB |
| B | Demineralized water | AQUA | A 100 |
| | EDTA | EDTA | 0.10 |
| | Glicerol | GLYCERIN | 3 |
| | Wetting DUB DIOL | METHYL PROPANEDYOL | 2 |
| | Active ingredient: | PANTHENOL | 0.5 |
| | Panthenol | | |
| | Active ingredient | BETAGLUCAN | 2 |
| | PLERASAN | | |
| | | CARBOMER | 0.40 |
| C | Vitamin E | TOCOPHERYL ACETATE | 0.5 |
| D | NAOH (SOL30%) | SODIUM HYDROXIDE | QB |
| E | Preservatives | | QB |
| | Perfume | PARFUM | 0.3 |

Preparation

1. mix all components of phase (A), heat at 75° C. under stirring

2. prepare phase (B) heating at 75° C.; the components are added in the order indicated and the turboemulsifier is activated for 30 seconds;

3. add phase (A) to phase (B) and cool with the turbine at slow speed to 45° C.;

4. add phase (C) under turbine mixing for 30 seconds;

5. add phase (D) and mix with turbine for 30 seconds;

6. cool under mild stirring to room temperature, then add phase (E) up to pH 5.5-6.0.

7—Lip Gloss—Ref. 0073

| Phase | Components | INCI NAME | % |
|---|---|---|---|
| A | DUB PTIS ester | PENTAERYTHRYTYL | 19.40 |
| | | TETRAISOSTEARATE | |
| | OLIPHYL 7525 | Olea Europaea Diglycerides and | 17.20 |
| | | triglycerides | |
| | BW WAX ESTER BW 67 | STEARYL BEESWAX, | 4.0 |
| | | BEHENYL BEESWAX | |
| | SILICA AREOSIL R972 | SILICA DIMETHYL SILILATE | 4.0 |
| B | Polymer INDOPOL H 300 | POLYBUTENE | 47.90 |
| | Mica AMIPEARL 4503 | CI 77491: Mica | 1.5 |
| | Mica AMIPEARL 4507 | CI 77491: Mica | 1.5 |
| | Acrylic Polymer | POLYMETHYL | 3.0 |
| | JURIMER | METHACRYLATE | |
| | Preservative | PHENOXYETHANOL, | 0.5 |
| | ISOCIDE PI | METHYLPARABEN, | |
| | | PROPYLPARABEN, | |
| | | BUTYLPARABEN, ETHYLPARABEN | |
| B′ | Perfume | PARFUM | 1.0 |

Preparation

1. mix all components of phase (A), heat at 80° C. under stirring; disperse silica

2. add slowly, in the order indicated, the ingredients of phase (B), under constant mixing at 80° C.

3. cool slowly to 60° C., add phase (B′) then pour in the final container.

8—Suncare Oil—Ref. 0056

| Phase | Components | INCI NAME | % |
|---|---|---|---|
| A | Lipophilic ester | C12C15 ALKYL BENZOATE | 5 |
| | DUB B1215 | | |
| | OLIPHYL 7525 | Olea Europaea Diglycerides and | 40 |
| | | triglycerides | |
| | Saturated oil | C15-19 ALKANE | 10 |
| | GEMSEAL 40 | | |
| | Saturated oil | ISOHEXADECANE | 5 |
| | ARLAMOL HD | | |
| | Octyl dodecanol | OCTYLDODECANOL | QB A 100 |
| | EUTANOL G | | |
| | Lipophilic ester | ISODECYL NEOPENTANOATE | 3 |
| | DUB VCI 10 | | |
| | Sunscreen | ETHYLHEXYLMETHOXYCINNAMATE | 4 |
| | NEO HELIOPAN AV | | |
| | sunscreen | BUTYLMETHOXYDIBENZOYL | 0.5 |
| | UVUNUL BMDM | METHANE | |
| | BURITI REF oil | MAURITIA FLEXUOSA (FRUIT) | 2 |
| | | OIL | |
| | Lemon essential oil | CITRUS LIMONUM | 0.2 |
| | Vitamin E | TOCOPHERYL ACETATE | 0.3 |

Preparation

1. mix all components of phase (A) with vigorous stirring for 10-15 min, heating at 35-40° C.

9—Lips Butter—Ref.0059

| Fase | Componenti | Denominazione INCI | % |
|---|---|---|---|
| A | Castor oil | RICINUS COMMUNIS SEED OIL | 30 |
| | OLIPHYL 7525 | Olea Europaea Diglycerides and | 15.40 |
| | | triglycerides | |
| | LIPOPHYL ARGAN oil | ARGAN OIL | 8 |
| | Olive oil | OLEA EUROPAEA OIL | 10 |
| B | CUPUAçU butter | THEOBROMA GRANDIFLORUM | 12 |
| | | (SEED BUTTER) | |
| | White beeswax | CERA ALBA | 13 |
| | ABWAX WHITE BEESWAX | | |
| | PEARL FU | | |
| | Cacao butter | THEOBROMA CACAO | 0.50 |
| | ABWAX CANDELILLA slides | CERA CANDELILLA | 5 |
| | ABWAX CARNAUBA T1 | CERA CARNAUBA | 5 |
| | flakes | | |
| C | Propolis liposoluble extract | PROPOLIS, SOYA HYSPIDA | 1 |
| | APEROXID TLA | Tocopherol, Lecithin, Ascorbyl | 0.1 |
| | | Palmitate, Citric Acid | |

Preparation

1. mix all components of phase (A) in the order indicated, heat at 75° C. under stirring;

2. add waxes of phase (B) to phase (A), under stirring at 80° C.

3. add phase (C) pre-mixed with phase (A+B), mixing at 80° C. for 5-10 minutes until the dispersion is homogeneous;

4. pour into molds at 80° C., then cool.

10.—Restructuring Shampoo—Ref. 0091

| Phase | Components | INCI name | % |
|---|---|---|---|
| A | Surface-active | Sodium Trideceth Sulfate (and) | 40 |
| | substance | Sodium Lauroamphoacetate | |
| | MIRACARE | (and) Cocamide MIPA (and) | |
| | SLB/365-G | Water | |
| B | Demineralized water | AQUA | A 100 |
| | EDTA | EDTA | 0.15 |
| | Thickening agent | Hydrohypropyl Guar | 0.30 |
| | | Hydroxypropyltrimonium | |
| | | Chloride | |
| C | OLIPHYL 7525 | Olea Europaea Diglycerides | 15 |
| | | and triglycerides | |
| | Antioxidant | | QB |
| | Flavour | Parfum | 0.6 |
| D | Preservative | . . . | QB |
| E | Salt | Sodium Chloride | 2 |
| F | Acidity corrector | CITRIC ACID | QB a pH |
| | | | 5.5-5.8 |

Preparation

1. weigh phase (B) and add the same to phase (B);

2. add, in the order, phases (C), (D), (E) and (F), controlling the pH and viscosity and 3.3. adjusting same with phases (E) and (F).

11—Face Cream—Ref. 0066

| Phase | Components | INCI name | % |
|---|---|---|---|
| A | Emulsifier | SUCROSE TRISTEARATE | 0.5 |
| | DUB SE 3S | | |
| | cetearyl alcohol | CETEARYL ALCOHOL | 0.5 |
| | OLIPHYL 7525 | Olea Europaea Diglycerides | 15.0 |
| | | and triglycerides | |
| | KARITE' butter | BUTYROSPERMUM PARKII | 3.0 |
| | JOJOBA oil | SIMMONDSIA CHINENSIS | 0.5 |
| | Antioxidant | | QB |
| B | Demineralized water | AQUA | q.b. 100 |
| | Sequestrant: EDTA | EDTA | 0.10 |
| | Glycerol | GLYCERIN | 2.0 |
| | Active ingredient | PANTHENOL | 0.5 |
| | VITAPHYL | | |
| | PANTHENOL | | |
| | Allantoina | ALLANTOIN | 0.2 |
| | POLACRIL 41 polymer | CARBOMER | 0.15 |
| | POLACRIL 40 polymer | CARBOMER | 0.15 |
| | DUB SE 15P | SUCROSE PALMITATE | 2.5 |
| C | Vitamin E | TOCOPHERYL ACETATE | 0.5 |
| D | NAOH (Solution) | SODIUM HYDROXYDE | q.b. |
| E | Preservatives | | q.b. |
| | Perfume | PARFUM | 0.3 |

Preparation

1. mix all components of phase (A), heat at 75° C. under stirring;

2. prepare phase (B) heating at 75° C.; the components should be added in the order indicated; activate the turbine for 30 seconds;

3. add phase (A) into phase (B) and cool at 45° C., with slow turbine;

4. add phase (C) with active turbine for 30″;

5. neutralize phase (D) and activate turbine until the dispersion is complete;

6. cool with slow stirring to room temperature, then add phase (E), adjust the pH to 5.5-6.0 if needed.

12—Lip Stick—Ref.0055

| Phase | Components | INCI name | % |
|---|---|---|---|
| A | Polyethylene | POLYETHYLENE | 5.00 |
| | ABWAX microcrystalline wax | MICROCRYSTALLINE WAX | 1.00 |
| | DUB ISIS ester | ISOSTEARYL ISOSTEARATE | 10.00 |
| | GEMSEAL 40 saturated oil | C15-19 ALKANE | 8.00 |
| | OLIPHYL 7525 | Olea Europaea Diglycerides | 20.00 |
| | | and triglycerides | |
| | DUB SSIC ester | ISOCETYL STEAROYL | 10.00 |
| | | STEARATE | |
| | Polybutene | POLYBUTENE | 6.00 |
| | VP/HEXADECENE | VP/HEXADECENE | 3.00 |
| | COPOLYMER | COPOLYMER | |
| B | PHENYLPROPYLDIMETHYL | PHENYLPROPYLDIMETHYL | 2.00 |
| | SILOXYSILICATE | SILOXYSILICATE | |
| | ABWAX white beeswax | BEESWAX | 4.00 |
| | ABWAX Candelilla wax | CERA CANDELILLA | 4.00 |
| | LIPOPHYL CUPUAçU BUTTER | THEOBROMA | 1.00 |
| | | GRANDIFLORUM | |
| | | (SEED BUTTER) | |
| C | Preservatives | | q.s. |
| | ISOCID BBE | GLYCINE SOYA, BHT, | 0.20 |
| | | BHA,TOCOPHERYL ACETATE | |
| D | RED 7/CASTOR OIL | C.I. 77850/RICINUS | 5.00 |
| | | COMMUNIS SEED OIL | |
| | WHITE/CASTOR OIL | C.I. 77891/RICINUS | 5.00 |
| | | COMMUNIS SEED OIL | |
| | RED FE OXIDE/CASTOR OIL | C.I. 77491/RICINUS | 6.00 |
| | | COMMUNIS SEED OIL | |
| | BLUE/CASTOR OIL | C.I. 77007/RICINUS | 1.00 |
| | | COMMUNIS SEED OIL | |
| | YELLOW FE OXIDE/CASTOR | C.I. 77492/RICINUS | 1.00 |
| | OIL | COMMUNIS SEED OIL | |
| E | Polyurethane | POLYURETHANE | 2.00 |
| | ALUMINUM STARCH | ALUMINUM STARCH | 1.50 |
| | OCTENYLSUCCINATE | OCTENYLSUCCINATE | |
| | Mica | MICA | 1.50 |
| F | Aroma | AROMA | QB |
| | LIPOPHYL BURITI oil | MAURITIA FLEXUOSA | 0.10 |
| | | (FRUIT) OIL | |

Preparation

1. mix the components of phase (A) in the order indicated, dissolving waxes at 115-120° C.;

2. prepare separately phase (B), adding the components in the indicated order and heating at 80° C., then add phase (A);

3. add pre-mixed phases (C) and (D) to phase (B), mix at 80° C. until the pigment is homogenously dispersed;

4. add the powders of phase (E) to the bulk; add phase (F) and pour in the molds.

13—Eyelashes Elongating Mascara—Ref. 0031

| Phase | Components | INCI name | % |
|---|---|---|---|
| A | OLIPHYL 7525 | Olea Europaea Diglycerides | 20.00 |
| | | and triglycerides | |
| | CANDELILLA wax | CERA CANDELILLA | 8.00 |
| | BELLINA wax | Polyglyceryl 3-beeswax | 3.00 |
| | ANTARON V220 | VP/EICOSENE COPOLYMER | 5.00 |
| | Trimethylsiloxysilicate | TRIMETHYLSILOXYLICATE | 10.00 |
| A′ | WP black Pigment | PIGMENTO NERO WP | 10.00 |
| B | Demineralized water | Aqua | q.b. |
| | | | a 100 |
| | Preservatives | . . . | q.b |

Preparation

1. mix all components of phase (A) with strong stirring, heating at 80° C.;

2. disperse the black pigment maintaining the temperature at 80° C.;

3. pour slowly, with vigorous stirring, phase (B) into phase (A+A′), then cool to room temperature.

14—Massage Oil—Ref. 0008

| Phase | Components | INCI name | % |
|---|---|---|---|
| A | DUB B1215 oil | C12C15 ALKYL BENZOATE | 20 |
| | OLIPHYL 7525 | Olea Europaea Diglycerides and | 39.0 |
| | | triglycerides | |
| | EUTANOL G octyl- | OCTYLDODECANOL | q.b. a |
| | dodecanol | | 100 |
| | MIRASIL CM 5 silicone | CYCLOMETHICONE | 20 |
| | NEXBASE 2004 oil | POLYDECENE | 5 |
| | Raffinated BURITI oil | MAURITIA FLEXUOSA | 0.5 |
| | | (FRUIT) OIL | |
| | essential oil-Perfume | Perfume | 0.5-2 |

Preparation

1. mix all components of phase (A) with vigorous stirring for 10-15 min, heating at 35-40° C.

1. A process for the preparation of a mixture of fatty acids glycerol esters suitable for cosmetic and pharmaceutical applications, which comprises the following steps:
a) neutralization of a vegetal oil with NaOH, with consequent formation of soap pastes;
b) acidic hydrolysis of the soap pastes, with consequent formation of a fatty phase, containing free fatty acids or fatty acid glycerol esters and an aqueous phase containing mucilages, impurities and phospholipids;
c) separation of the fatty acids from the liquid phase;
d) distillation of the fatty acids at a pressure from 1 to 3 mbar;
e) esterification of the distilled fatty acids with glycerine, in a glycerine/fatty acids weight ratio of 1-2:10, at a temperature between 200 and 220° C. and a pressure between 1 and 3 mbar;
f) neutralization of the obtained product to eliminate the residual acidity;
g) decolouring, deodorising and optional winterization of the product by means of addition of direct vapour at a pressure from 1 to 3 mbar.

2. The process according to claim 1, further comprising the hydrolysis of the undistilled residual fraction at step d) containing neutral oil, with consequent formation of fatty acids and an aqueous phase containing glycerine, followed by separation of the aqueous phase from the fatty acids and processing of the latter according to steps d)-g).

3. The process according to claim 1, wherein the aqueous phase containing glycerine is concentrated, centrifuged and then distilled under deep vacuum, preferably at 1-4 mbar, at a temperature of 140-170° C., to obtain high purity glycerine, which is used for the esterification of the fatty acids at step e).

4. The process according to claim 1, wherein the neutralization of step a) is carried out with NaOH at 20 Bè.

5. The process according to claim 1, wherein the fatty acids distilled at step d) have a purity of at least 99.5%.

6. The process according to claim 2, wherein the hydrolysis of the residual fraction is carried out at a pressure of 30 bar and temperature of 230° C.

7. The process according to claim 3, wherein the distilled glycerine has a purity of at least 99.5%.

8. The process according to claim 1, wherein the product obtained at the end of step g) is subjected to molecular distillation at a temperature between 220 and 280° C., operating at a maximal pressure of 0.001 mbar through a thin film evaporator and/or short path distillator, with an average residence time of the product between 30 sec and 2 minutes, to prepare distilled fractions enriched in diglycerides with purity exceeding 90% or triglyceride residual fractions with purity exceeding 95%.

9. The process according to claim 1, wherein the vegetal oil used at step a) is selected from olive oil, sunflower oil and rapeseed oil.

10. The process according to claim 9, wherein olive oil is used.

11. A mixture of fatty acids glycerol esters obtainable by the process of claim 1.

12. The mixture according to claim 11, comprising the following composition % of fatty acids, when olive oil and sunflower oil are used as the starting materials:

| | Olive Oil | Sunflower Oil |
|---|---|---|
| Palmitic | 5-12% | 5-7.6% |
| Palmitoleic | 1.5-2.5% | 0-0.3% |
| Stearic | 1.5-4% | 2.7-6.5% |
| Oleic | 65-80% | 14.0-39.4% |
| Elaidinic (trans-oleic C18 n-9) | 0-0.4% | — |
| and petroselinic (cis C18 n-12) | | |
| Linoleic | 10-20% | 48.3-74.0% |
| Linolenic | 0.5-1% | 0-0.3% |
| Arachic | 0.3-0.7% | 0.1-0.6% |
| Eicosenoic (gadoleic) | 0.2-0.6% | 0-0.3% |
| Beenic | 0.1-0.3% | 0.3-1.5% |
| Erucic | — | 0-0.1% |
| Lignoceric | 0.1-0.3% | 0-0.5% |
| Miristic | — | 0-0.2% |
| Lauric | — | 0-0.1% |
| Heptadecanoic | — | 0-0.2% |
| Heptadecenoic | — | 0-0.1% |

13. The mixture according to claim 11 containing from 25 to 75% triglycerides, from 0 to 50% diglycerides and from 0 to 25% monoglycerides.

14. A cosmetic or pharmaceutical composition containing a mixture of fatty acids glycerol esters according to claim 11.

15. The cosmetic composition according to the claim 14, which is in the form of a cream, gel, unguent, lotion, emulsion O/W and W/O, detergent, shampoo, bath foam, gel and shower foam, liquid foam, stick, make up, cosmetic oil.

16. A method for the cosmetic treatment of skin, hair, eyes contour, lips, which comprises the application of a composition according to claim 14 on the interested body part.

17. A method of preparing a cosmetic or pharmaceutical composition comprising combining with an active ingredient a mixture of fatty acids glycerol esters according to claim 11, as a vehicle or solubiliser of active ingredients, or as excipient.
